(19) United States
(12) Patent Application Publication
(10) Pub. No.: US 2024/0389992 A1
(43) Pub. Date: Nov. 28, 2024

(54) Surgical Retractor

(71) Applicant: CLARIANCE, BEAURAINS (FR)

(72) Inventor: Brice Krier, St Leger (FR)

(21) Appl. No.: 18/274,480

(22) PCT Filed: Feb. 1, 2022
(86) PCT No.: PCT/FR22/50177

§ 371 Date: Jul. 14, 2024

Related U.S. Application Data
(60) Provisional application No. 63/144,156, filed on Feb. 1, 2021.

(30) Foreign Application Priority Data
Apr. 2, 2021 (FR) FR2103425

(51) Int. Cl. A61B 17/02; A61B 17/00

(52) U.S. Cl. CPC A61B 17/02; A61B 2017/00367; A61B 2017/00915

(57) ABSTRACT

The invention relates to a surgical retractor (E) comprising: —a frame (100) having a central opening (110), —arms (210, 220, 230) movable in translation, —a blade (310, 320, 330) associated with each arm, characterized in that it comprises two independent wheel portions (410, 420) guided in rotation by the frame (100) and collaborating with different arms (210, 220, 230) in order to provide independent movements.

### FIELD OF APPLICATION OF THE INVENTION

The invention relates to the field of surgical retractors, and in particular, to adaptations making it possible to improve the functionalities of said retractors.

### DESCRIPTION OF THE PRIOR ART

In the field of surgery, in particular mini-invasive, it is known to use surgical retractors which ensure the retraction of tissue of the patient to propose a lesionless access for the practitioner.

Among surgical retractors, there are retractors adopting a concentric geometry comprising movable blades (also called valves) moving concentrically, the retraction of which together creates the access path.

Among these retractors, document WO0103586 describes a retractor device with extendable needles or blades, comprising a base designed for the purpose of positioning over an opening made in a body part, and an extendable needle-shaped retractor element comprising a longitudinal axis provided with a opening, the configuration of which enables the insertion via the opening, so as to penetrate the body part and thus engage a body tissue. The retractor element has a first supported end, associated with the base and a second free end, axially separated from the base. An expansion apparatus, mounted on the base, makes it possible to radially deploy the retractor element, adjustably about the longitudinal axis, in order to retract a tissue engaged by the retractor element, thus making it possible to clear a working zone inside the body part, in generally axial alignment with the opening.

The base thus serves as a support and for guiding a drive wheel controlled by the user. By a transmission means, the rotation of the wheel causes the radial translation of a plurality of arms. Each arm is associated with a needle, such that the rotation in a direction or in the other of the drive wheel causes the retraction or the synchronised alignment of the blades.

Such a retractor is bulky. In addition, controlling the rotation of the wheel is done by means of at least one rotary button disposed on the base. Consequently, the visual accessibility is reduced when the surgeon actuates the button.

Furthermore, the rotation of the button must ensure the rotation of the wheel, the rotation movement of which must be transformed by a thumbwheel system in translation movement for the arms. Such a device therefore requires complex transmission means multiplying the number of constitutive elements.

There are also, in the prior art, retractors with concentric geometry which enable the desynchronised movement of the arms. The implementation of such a function thus requires clutch means and separate control means, which complexify and make the retractor more bulky.

### BRIEF DESCRIPTION OF THE INVENTION

What has been observed, is that the applicant has carried out research aiming to improve surgical retractors.

Research has resulted in designing and producing a new, more ergonomic, less bulky surgical retractor, which has new functionalities.

This surgical retractor comprises:
a frame having a central opening,
arms which are movable in translation,
a blade associated with each arm,

The arms having two ends:

A first end engaging with the frame for guiding and driving purposes, and a second end operating in the central opening;

The blades having two ends:

A first end connected to the second end of an arm and a second end intended to engage with the tissue of the patient.

According to the invention, the retractor is noteworthy in that it comprises two independent wheel portions guided in rotation by the frame and engaging with different arms to propose independent movements.

This feature is particularly advantageous in that at least one arm (and therefore one blade) can be controlled separately. The separate movement is done by the fact that there is no longer only one moving wheel, but two separate wheel portions. It is no longer necessary to use a complex clutch means to enable these non-synchronised movements.

A favoured method of use of this retractor is for spine surgery. In particular, it enables access to the spine by retracting the tissue and the viscera, for example, laterally or anteriorly.

During its use, the retractor can be linked to an articulated surgical arm such as the so-called Martin's arm by means of its frame or by means of one of its arms.

To do this, according to another particularly advantageous feature of the invention, the frame is equipped with a mechanical connection means preformed from a fixing orifice enabling a fixing to said arm.

Likewise, according to another particularly advantageous feature of the invention, the first end of at least one arm is equipped with an orifice remaining accessible in any position (even retracted) enabling the connection with a surgical arm.

The separate blade can be fixed and/or supported on the body of the patient and it is the retractor which is moved with respect to the blade. It can also not be fixed or not supported. It is thus moved with respect to the frame of the retractor.

According to another particularly advantageous feature of the invention, the two wheel portions are not coaxial. This feature makes it possible to propose a less bulky frame, and therefore a less bulky retractor. Indeed, concentric wheel portions would overlap, which would have had the effect of increasing the thickness of the retractor, which is not the case with non-concentric wheel portions.

The wheel portions can transmit the movement by any means, for example, by adherence. However, according to a preferred, but non-limiting embodiment, the wheel portions are gearwheel portions.

According to a preferred, but non-limiting embodiment, there are three arms and associated blades disposed at 120 degrees about the axis of the cylinder formed by the blades grouped in a retracted position.

According to another particularly advantageous feature of the invention, each wheel portion is equipped with a movable control finger projecting outside of the frame, finger movable by the user. The movement fingers make it possible to have the same function as a thumbwheel that can be found in other devices, but by limiting the number of parts therefore increasing the robustness of the system.

The ergonomy is improved, as the induced movement of the finger enables more movement speed of the blades for the surgeon. In addition, the movement is intuitive, as a translation of the finger induces a blade translation and the return of the force is more direct.

According to another particularly advantageous feature, a fixed finger is fixed to the frame facing the stroke of each movable finger. This arrangement gives a support to the fingers of the user of the movable fingers, and makes it possible to reproduce for the user, the movements of the hand performed during the use of a clamp which further optimises the ergonomy.

The fact of having movable fingers makes it possible to offset the action of the hands of the user and to position them outside of the frame and therefore in a position far from the wound. This position further makes it possible to keep a better vision of the operating zone.

According to another particularly advantageous feature of the invention, the retractor further comprises a means for holding the blades in position.

To implement this function, each movable finger is associated with a cursor which has a pawl behaviour by enabling the rotation of the gearwheel portion in one direction (the direction corresponding to the retraction of the blades) and by blocking the rotation in another direction. A disengagement of the pawl controlled by the user enables the rotation in the opposite direction and therefore the retraction of the blades.

According to a preferred, but non-limiting embodiment, the cursor linked to the movable finger is mounted on a spring blade and comprises at least one tip which engages with oriented gearings associated with the fixed frame.

According to another particularly advantageous feature of the invention, the first end of the blades is preformed so as to house a functional module.

To do this, according to another feature, the first end of the blades is preformed with at least one orifice opening onto the internal surface of the blade.

This orifice forms a channel giving access to the retracted zone.

For example, it constitutes a light shaft when the blade houses a functional module of the light means type.

It also enables access to video means when the blade houses an endoscopic camera.

According to another particularly advantageous feature of the invention, at least the upper parts of the longitudinal edges of the blades have complementary interlocking profiles optimising the connection together once in contact.

According to another particularly advantageous feature of the invention, the blades are available in different lengths. The retractor is associated with a plurality of blade sets to form a kit. Thus, the surgeon has the option of selecting the blades which are the most suitable for the morphology of the patient operated on.

According to another particularly advantageous feature of the invention, the blades are made of a radiotransparent material.

According to another particularly advantageous feature of the invention, the blades are made of titanium and their thickness is refined in order to make them radiotransparent.

According to another particularly advantageous feature of the invention, the connection between the first end of the blades and the second end of the arms enables an inclination. The blades can be inclined (for example, up to fifteen degrees).

To do this, the blade is associated with a plate pivotingly mounted with respect to the arm. The plate therefore ensures an intermediate connecting part function between the arm and the blade.

According to a non-limiting embodiment, outside of the blades, the other constitutive elements are made of materials chosen from the following non-exhaustive list:
titanium alloy,
stainless steel,
any type of polymer,
aluminium or aluminium alloy.

The fundamental concepts of the invention have just been described above in their most basic form, other details and features will emerge more clearly upon reading the description below and regarding the accompanying drawings, giving as a non-limiting example, an embodiment of a retractor according to the invention.

### BRIEF DESCRIPTION OF THE DRAWINGS

FIG. 1 is a schematic drawing of a top view of an embodiment of a retractor according to the invention in the retracted position;

FIG. 2 is a schematic drawing of a top view of the retractor of FIG. 1 in the retracted position;

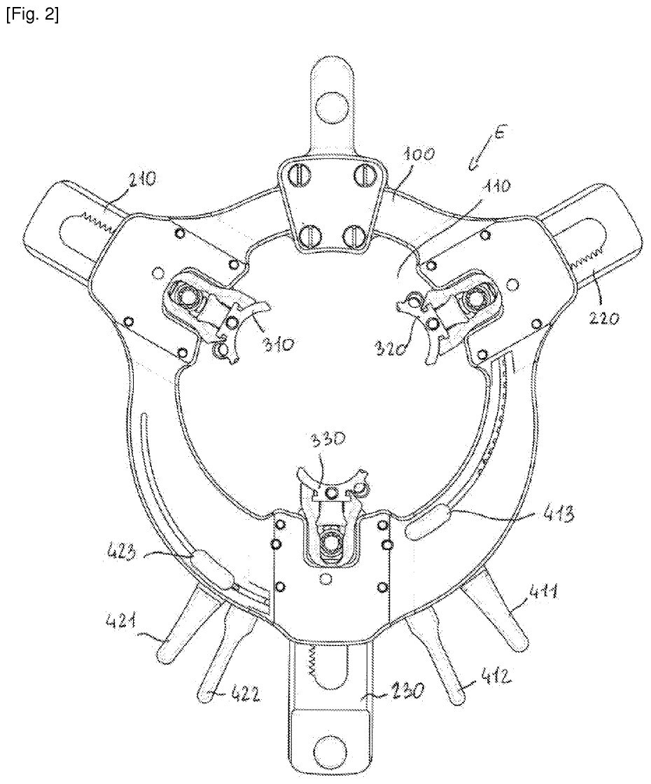

FIG. 3 is a schematic drawing of a top view of the retractor of FIG. 1 with different blade positions;

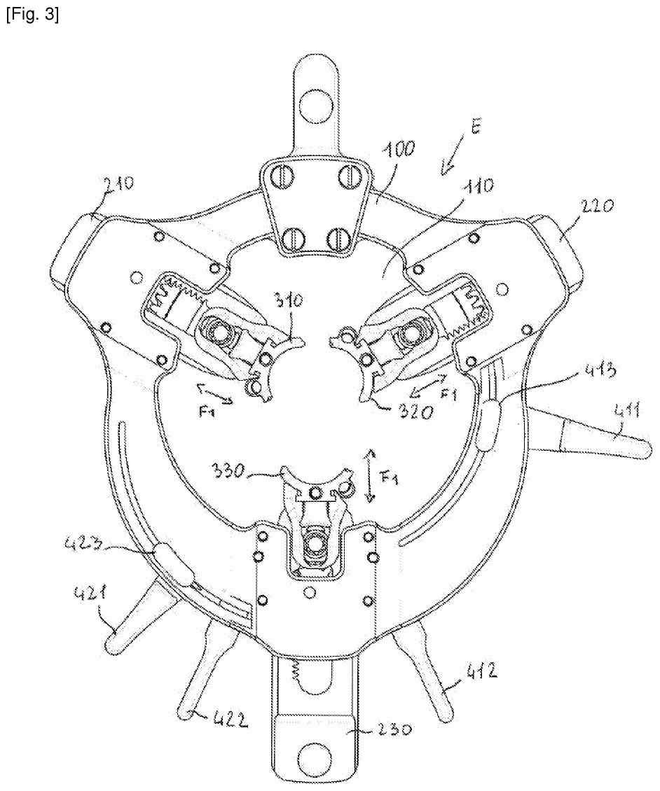

FIG. 4 is a schematic drawing of a partially exploded top view of the retractor of FIG. 1;

FIG. 5 is a schematic drawing of a top view of the only frame of the retractor of FIG. 1;

FIG. 6 is a schematic drawing of a side view of the retractor of FIG. 1 in the retracted position;

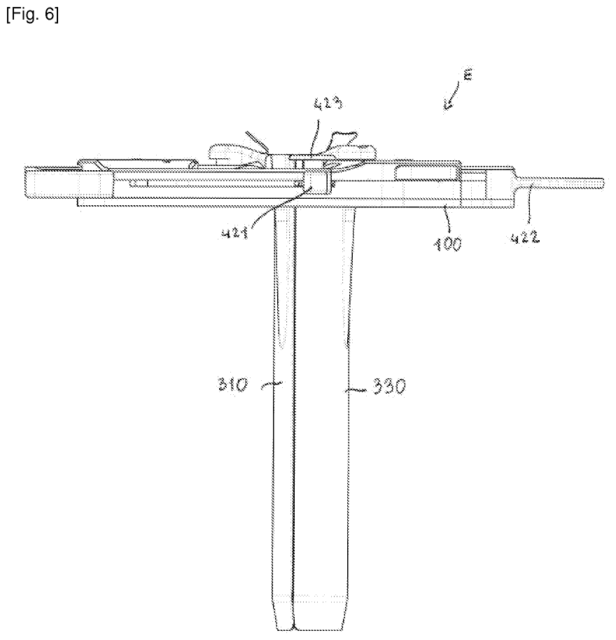

FIG. 7 is a schematic drawing of a perspective view of the detail of the connection between the first end of a blade and the second end of an arm;

FIG. 8 is a schematic drawing of a cross-sectional view of the retractor of FIG. 1 at one of the cursors equipping the retractor.

### DESCRIPTION OF A PREFERRED EMBODIMENT

As illustrated by the figures, the surgical retractor referenced E in its entirety comprises a frame 100 adopting a substantially annular geometry and having a central opening 110.

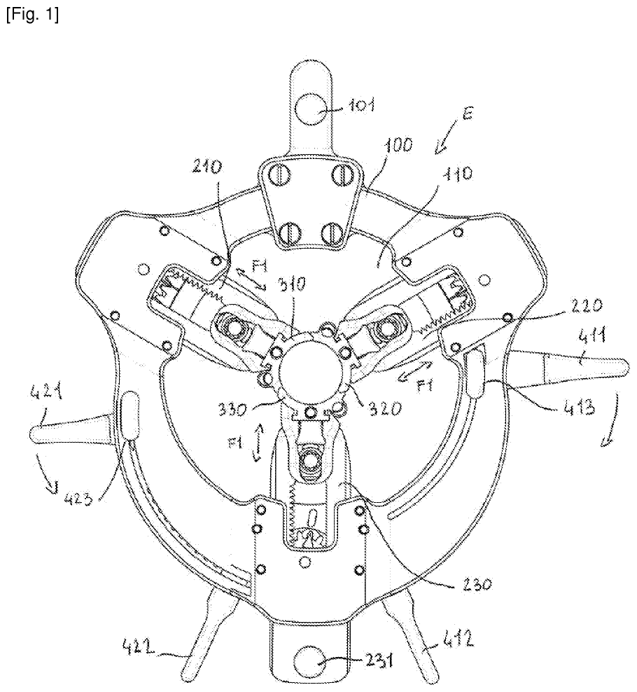

This frame 100 supports three arms 210, 220, 230 which are movable in translation (according to the double arrow F1) and disposed at 120 degrees on said frame 100. A blade 310, 320, 330 is associated with each arm. The three blades are identical and once in contact (see FIG. 1) complement one another to form a cylinder.

More specifically, according to the embodiment illustrated, at least the upper part of the longitudinal edges of the blades has a complementary interlocking profile optimising the connection together once in contact.

The arms 210, 220, 230 have two ends:
A first external end engaging with the frame 100 for guiding and driving purposes, and a second internal end operating in the central opening 110.

The blades 310, 320, 330 have two ends:
A first high end connected to the second internal end of an arm and a second low end intended to engage with the tissue of the patient.

During the implementation on the patient, the blades 310, 320, 330 are passed around dilators (not illustrated) previously inserted in the tissue of the patient. The length of the blades is chosen according to the graduations present on the last dilator.

The retractor E can be fixed on an operating table by means of a so-called Martin's surgical arm, not illustrated. The frame 100 is equipped with a mechanical connection means preformed with a fixing orifice 101 enabling a fixing to said arm.

As illustrated, the first end of at least one arm, in this case the arm 230, is equipped with an orifice 231 remaining accessible in any position (even retracted) and also enabling the connection with a surgical arm.

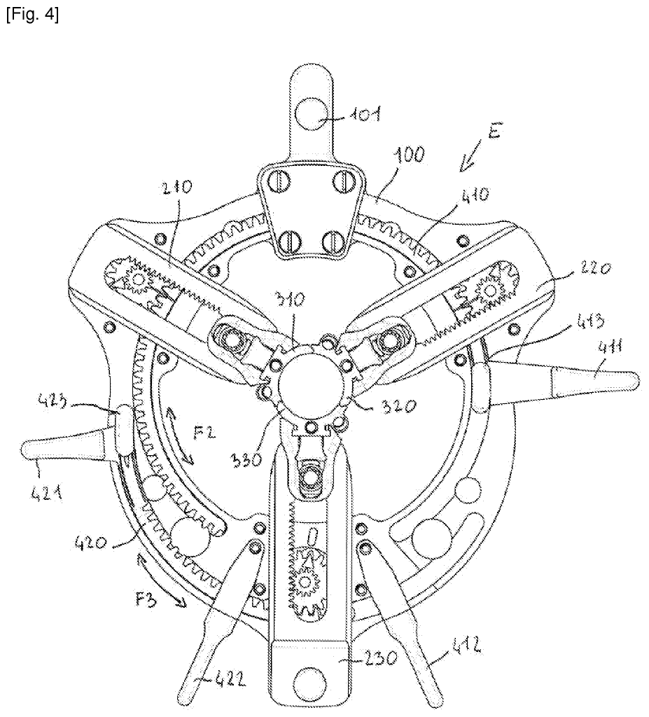

As illustrated in more detail in FIG. 4, the retractor E comprises two independent and non-coaxial gearwheel portions 410 and 420 guided in rotation by the frame 100 which is preformed for this purpose and engaging with different arms to propose independent movements.

Thus, the gearwheel portion 410 controls, by its rotation movement (double arrow F2), the translation (double arrow F1) of the arms 210 and 220.

The gearwheel portion 420 controls by its rotation movement (double arrow F3), the translation (double arrow F1) of the arm 230.

As illustrated, the gearwheel portions 410 and 420 are not concentric, but the axes of the horizontal translations according to the double arrow F1 are concurrent.

The separate control of one of the arms (in this case, the arm 230) enables a more flexible functioning of the retractor E. In particular, it makes it possible to consider a fixing of the retractor E on the body of the patient by way of the end of the blade 330.

According to the embodiment illustrated, each arm is slidingly mounted with respect to the frame 100 and is provided with an oblong longitudinal lumen performed on a side of a rack. This rack engages with a pinion of small coaxial diameter and integral in rotation with a pinion of larger diameter pivotingly mounted on the frame 100 and engaging with a gearwheel portion.

According to a preferred, but non-limiting embodiment, for an arm stroke of between 150 and 300 millimetres:
the gearwheel portions comprise between 30 and 50 teeth, and
the double pinions comprise on their small and large diameter, between 10 and 20 teeth.

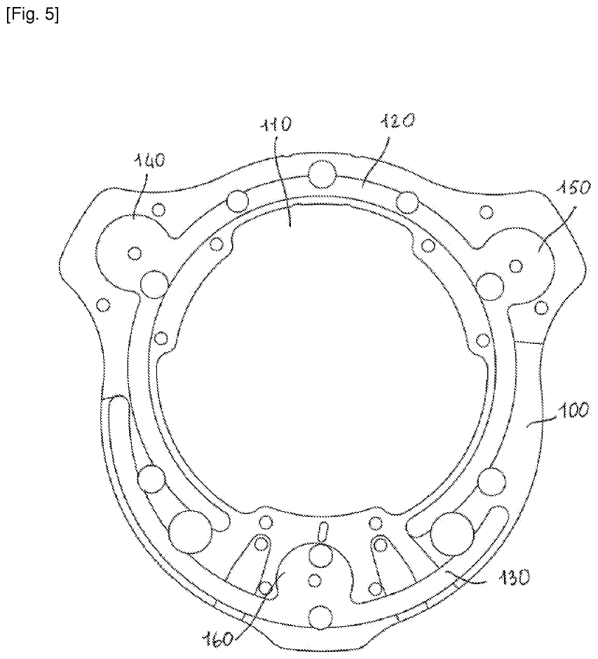

The frame 100 is formed of an upper part and of a lower part. FIG. 5 illustrates the lower part of the frame 100 preformed with grooves 120 and 130 guiding the rotation of the gearwheel portions respectively 410 and 420. The frame is also preformed with housings 140, 150 and 160 of the large-diameter pinions. The hollow volume of the groove 120 guiding the gearwheel portion 410 joins the hollow volume of the housings 140 and 150 intended to house the large-diameter pinions controlling the translation of the arms 210 and 220.

The hollow volume of the groove 130 guiding the gearwheel portion 420 joins the hollow volume of the housing 160 intended to house the large-diameter pinion controlling the translation of the independent arm 230.

As illustrated, the two gearwheel portions 410 and 420 operate in the same plane, which is made possible by their non-coaxiality. The frame and therefore the retractor E are thus compact and light.

As illustrated, each wheel portion 410 and 420 is equipped with a movable control finger 411 and 421 projecting in a direction radiating outside of the frame 100, finger movable by the user. A fixed finger 412, 422 is fixed to the frame 100 facing the stroke of each movable finger 411 and 421.

This fixed finger enables the user to press to actuate the movable finger. Thus, the user presses on the fixed finger 412 to actuate the movable finger 411 and control the translation of the arms 210 and 220. Likewise, they press on the fixed finger 422 to actuate the movable finger 421 and control the translation of the arm 230.

A cursor 413 and 423 is associated with each movable finger 411 and 421 and moves over the upper surface of the frame 100.

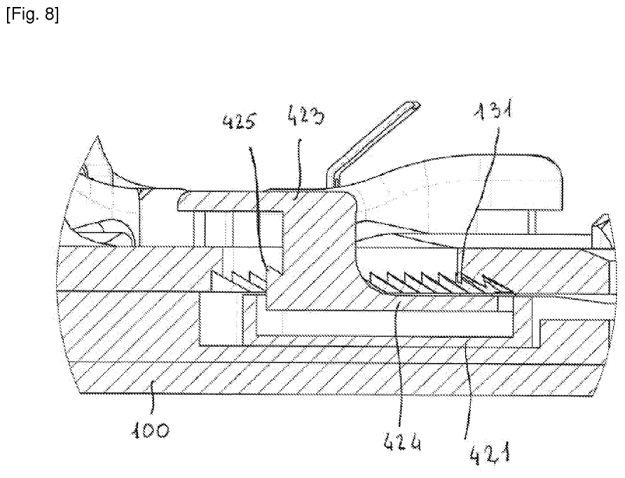

As illustrated by FIG. 8 for the cursor 423, it is mounted on a resilient blade 424 and is preformed to serve as a pawl, i.e. that it enables the rotation of the gearwheel portion to which it is associated in one single direction.

The user must press on this cursor 423 to enable the movement in the other direction. These cursors therefore serve as a means for holding the blades in position.

More specifically, an end of the movable cursor 423 is constituted of a bevelled tip 425 which operates in the frame 100. This tip 425 is in contact and is blocked in gearings 131 which are associated with the fixed frame 100.

The orientation of the gearings 131 enables the movement in a direction (corresponding to that of the retraction of the blades) and prevents the movement in the other (corresponding to the retraction).

Pressing on the cursor 423 causes a movement of the tip 425 enabled by the resilient blade 424, which ensures the disengagement between the tip 425 and the gearings 131.

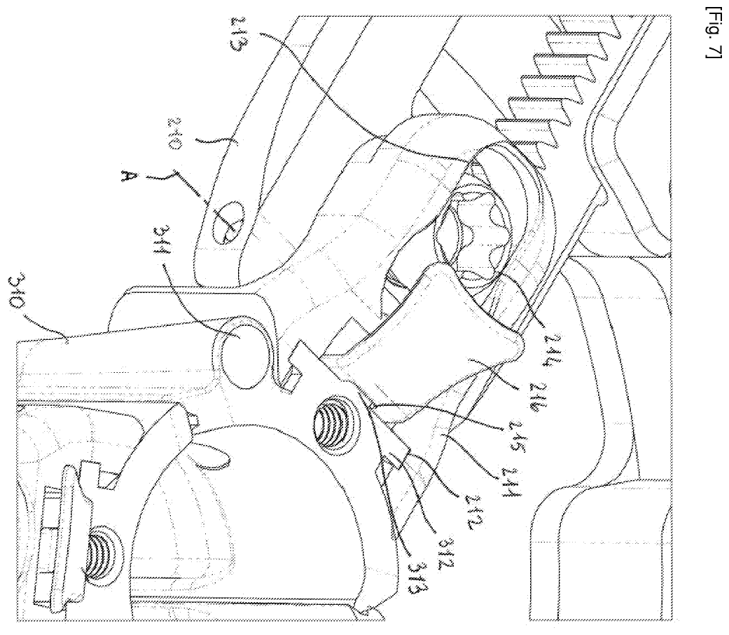

As illustrated by FIG. 7, the high end of the blades (in this case, the blade 310) is articulated to said arm 210 so as to enable a pivoting and the holding of an inclined position.

This functionality is, in this case, implemented by means of a plate 211 pivotingly mounted with respect to the second end of the arm 210 and comprising two ends:
a first end preformed of a groove 212 for housing a preformed rib 312 in the external surface of the blade 310,
a second end preformed of an oblong hole 213, wherein the head of a screw 214 screwed to the second end of the arm bears.

The screwing of the screw 214 controls the angular position of the plate and therefore of the blade with respect to the arm.

The pivot axis referenced A is perpendicular to the translation axis of the arm. Furthermore, this pivot axis is disposed in front of the action screw 214 which enables a fine angular movement of the plate 211 and therefore of the high end of the blade.

According to the preferred, but non-limiting embodiment illustrated, the rib 312 further comprises a lug 313. Thus lug 313 engages with a notch 215 made in a resilient strip 216 fixed to the plate 211 and ensuring the retaining of the blade 310 with respect to the plate 211. The elasticity of the strip 216 holds the retaining position, enables without action, the housing of the blade 310 in the groove 212 and requiring an action of the user on the strip 216 to dismount the blade 310.

This high end of the blades is further designed so as to house functional modules, by being preformed in its thickness with at least one (in this case, one) orifice 311 to form a channel having an axis different from that of the blade, so as to open onto the internal face of the blade. This channel is arranged on a side of the blade.

The functional module can be different or not for each blade and is selected from the following list:
light means,
endoscopic camera,
etc.

What follows is a method for using the retractor E such as described and represented above. In this regard, it must be understood that the description below is a method from among a plurality of different methods, wherein such a retractor can be used. To use the retractor E, an incision is made in the body of a patient. A first dilator is then inserted in the incision to create a channel through the muscles. Other dilators of successively larger diameters can be inserted above the first dilator. The suitable blades 310, 320 and 330 are selected according to the penetration depth of the last dilator introduced. The blade 310 is assembled to the retractor E by sliding it and by clipping it in its housing groove 212. Similarly, the blades 320 and 330 are assembled to the retractor E by sliding them and by clipping them in their respective housing groove. The retractor E with the blades in a closed position and without angulation or inclination, is inserted in the operating site above the last dilator introduced, which is thus removed to create, through the muscles, a free-access channel to the spine. At this stage, the functional module(s) can be introduced in the orifices of the blades 310, 320 and 330 provided for this purpose. By pressing on the fixed finger 422, the movable finger 421 is actuated to control the translation of the arm 230 and of the blade 330. To retract the two arms 210 and 220 and the two blades 310 and 320, the movable finger 411 is actuated by pressing on the fixed finger 412. To increase the inclination of the blade 310, the screw 214 is screwed in the clockwise direction. To relax the inclination of the blade 310, the screw 214 is unscrewed in the anti-clockwise direction. Similarly, the inclination of the blades 320 and 330 can each be independently adjusted. In this way, the low ends of the blades can be retracted without significantly modifying the position of the high ends of the blades. And thus, an opening of a diameter, larger than that created by the high ends, is created in the proximity of the intervertebral space to be operated on.

It is understood that the retractor, which has just been described and represented above, has been so in view of a disclosure rather than a limitation. Naturally, various arrangements, modifications and improvements can be applied to the example above, without moving away from the scope of the invention.

1. Surgical retractor (E) comprising:
a frame (100) having a central opening (110),
arms (210, 220, 230) which are movable in translation, and
a blade (310, 320, 330) associated with each arm,
the arms (210, 220, 230) having two ends:
a first end engaging with the frame (100) for guiding and driving purposes and a second end operating in the central opening (110),
the blades (310, 320, 330) having two ends:
a first end connected to the second end of an arm (210, 220, 230) and a second end intended to engage with the tissue of the patient,
CHARACTERISED BY THE FACT THAT the retractor comprises two independent wheel portions (410, 420) guided in rotation by the frame (100) and engaging with different arms (210, 220 and 230) to propose independent movements.

2. The retractor (E) according to claim 1, CHARACTERISED BY THE FACT THAT the two wheel portions (410, 420) are not coaxial.

3. The retractor (E) according to claim 1, CHARACTERISED BY THE FACT THAT each wheel portion (410, 420) is equipped with a movable control finger (411, 421) projecting outside of the frame (100), finger movable by the user.

4. The retractor (E) according to claim 3, CHARACTERISED BY THE FACT THAT a fixed finger (412, 422) is fixed to the frame (100) facing a stroke of each movable finger (411, 421).

5. The retractor (E) according to claim 1, CHARACTERISED BY THE FACT THAT the retractor comprises a means (413, 423) for holding each wheel portion in position, adopting the behaviour of a pawl.

6. The retractor (E) according to claim 1, CHARACTERISED BY THE FACT THAT the first end of the blades (310, 320, 330) is preformed so as to house a functional module.

7. The retractor (E) according to claim 6, CHARACTERISED BY THE FACT THAT the first end of the blades (310, 320, 330) is preformed with at least one orifice (311) opening onto the internal surface of the blade.

8. The retractor (E) according to claim 1, CHARACTERISED BY THE FACT THAT the wheel portions (410, 420) are gearwheel portions.

9. The retractor (E) according to claim 8, CHARACTERISED BY THE FACT THAT each arm (210, 220, 230) is slidingly mounted with respect to the frame (100) and is provided with an oblong longitudinal lumen preformed on a side of a rack, this rack engages with a pinion of small coaxial diameter and integral in rotation with a pinion of a larger diameter pivotingly mounted on the frame (100) and engaging with a gearwheel portion.

10. The retractor (E) according to claim 1, CHARACTERISED BY THE FACT THAT the connection between the first end of the blades (310, 320, 330) and the second end of the arms (210, 220, 230) enables an inclination.

11. The retractor (E) according to claim 1, CHARACTERISED BY THE FACT THAT at least upper parts of the longitudinal edges of the blades (310, 320, 330) have complementary interlocking profiles optimising the connection together once in contact.

12. The retractor (E) according to claim 1, CHARACTERISED BY THE FACT THAT the blades (310, 320, 330) are made of a radiotransparent material.

13. The retractor (E) according to claim 1, CHARACTERISED BY THE FACT THAT the blades are made of titanium and the thickness of the blades (310, 320, 330) is refined in order to make the blades radiotransparent.
